(19) United States
(12) Patent Application Publication
(10) Pub. No.: US 2008/0208015 A1
(43) Pub. Date: Aug. 28, 2008

(54) SYSTEM, APPARATUS AND METHOD FOR REAL-TIME HEALTH FEEDBACK ON A MOBILE DEVICE BASED ON PHYSIOLOGICAL, CONTEXTUAL AND SELF-MONITORED INDICATORS OF MENTAL AND PHYSICAL HEALTH STATES

(76) Inventors: Margaret E. Morris, Portland, OR (US); Farzin Guilak, Beaverton, OR (US); Michael E. Labhard, Lake Oswego, OR (US); Mick J. Flanigan, Beaverton, OR (US)

Correspondence Address: INTEL CORPORATION;c/o INTELLEVATE, LLC, P.O. BOX 52050, MINNEAPOLIS, MN, 55402, US

(21) Appl. No.: 12/028,681

(22) Filed: Feb. 8, 2008

Related U.S. Application Data
(60) Provisional application No. 60/900,483, filed on Feb. 9, 2007.
(60) Provisional application No. 60/900,484, filed on Feb. 9, 2007.

(51) Int. Cl. A61B 5/02; A61B 5/01

(52) U.S. Cl. 600/301

(57) ABSTRACT

A system, apparatus and method for real-time health feedback on a mobile device based on physiological, contextual and self-monitored indicators of mental and physical health states. An embodiment of an apparatus is capable of receiving data relating to the physical or mental health states of a user from an input device and passive sensing. The apparatus is further capable of providing a mobile intervention via an output device and capable of measuring the effectiveness of the mobile intervention in improving the physical or mental health states and generating self-awareness. Other embodiments are described and claimed.

### CROSS-REFERENCE TO RELATED APPLICATIONS

This application is related to pending U.S. patent application Ser. No. 11/641,973, filed on Dec. 20, 2006, and entitled “Apparatus for Monitoring Physiological, Activity and Environmental Data,” by inventors Margaret Morris et al. This application is related to pending U.S. patent application Ser. No. 11/704,703, filed on Feb. 9, 2007, and entitled “System, Apparatus and Method for Emotional Experience Time Sampling via a Mobile Graphical User Interface,” by inventor Margaret Morris. This application is related to and claims priority to pending U.S. Provisional Patent Application No. 60/900,483, filed on Feb. 9, 2007, and entitled “System, Apparatus and Method for Real-Time Health Feedback on a Mobile Device Based on Physiological, Contextual and Self-Monitored Indicators of Mental and Physical Health States,” by inventors Margaret Morris et al. This application is related to and claims priority to pending U.S. Provisional Patent Application No. 60/900,484, filed on Feb. 9, 2007, and entitled “System, Apparatus and Method for Mobile Real-Time Feedback Based on Changes In Heart Rate Variability to Enhance Cognitive Behavioral Therapy for Anger or Stress Reduction,” by inventors Mick J. Flanigan et. al. This application is related to pending U.S. patent application Ser. No. ______, filed on ______, and entitled “System, Apparatus and Method for Mobile Real-Time Feedback Based on Changes in the Heart to Enhance Cognitive Behavioral Therapy for Anger or Stress Reduction,” by inventors Margaret E. Morris et al.

### BACKGROUND

The vast majority of medical care remains focused on late stage illness, a bias that perpetuates the health care crisis in the United States and internationally. It is estimated that seventy-five percent of national health care costs in the United States relate to the treatment of chronic diseases.

To a large extent, the diseases that we treat almost exclusively in their late stages progress predictably, as do the costs of treatment. Yet, symptoms of diseases that are difficult and costly to treat in late stages can be stabilized and sometimes reversed if addressed early.

Shifting more attention and resources to preventive care could certainly increase return on medical investment. But, there are significant barriers. The close monitoring of risk factors required for preventive medicine is difficult for both clinicians and individuals. Today, clinicians lack sensitive tools to determine individual baselines of premorbid functioning and early signs of decline. Individuals frequently deny illness and avoid clinical assessment until a disease is in its later stages.

This application explores mind-body interventions on emerging technologies for preventive care.

### BRIEF DESCRIPTION OF THE DRAWINGS

FIG. 1 illustrates one embodiment of a system for mobile real-time health feedback based on physiological, contextual and/or self-monitored indicator(s).

FIG. 2 illustrates one embodiment of an apparatus for mobile real-time health feedback based on based on physiological, contextual and/or self-monitored indicator(s).

FIG. 3 illustrates one embodiment of a mobile feedback module.

FIG. 4 illustrates an example mobile therapy or intervention utilized by the present invention.

FIG. 5 illustrates an example mobile therapy or intervention utilized by the present invention.

FIG. 6 illustrates an example mobile therapy or intervention utilized by the present invention.

FIG. 7 illustrates one embodiment of a logic flow for mobile real-time feedback based on based on physiological, contextual and/or self-monitored indicator(s).

FIG. 8 illustrates the dynamic relationship between continuous monitoring and feedback in the embedded assessment approach.

### DETAILED DESCRIPTION

Emotional health is intimately intertwined with physical health. Psychological and behavioral interventions can help people modulate interpersonal stress and its consequences.

Some limitations in current behavioral medicine and psychotherapy include scalability, the stigma associated with psychotherapy, and lack of interventions at the point of need. Current resources are unable to provide good mental health care for everyone who might benefit from attention to mental health and/or psychological issues. People who might find benefit from behavioral or psychotherapy avoid seeking it because of perceived stigmas attached to mental disfunction. Further, current interventions are not available when and where they are most needed. For example, current interventions are currently provided in scheduled appointments and clinical settings, both of which are removed from the source of stress. Emerging technologies that emphasize mind-body relationships and preventive health care can bridge the divide.

Embedded assessment is a technology design strategy to drive preventive health care and early disease detection. In embedded assessment techniques, devices for collecting biometric data and assessing an individual's emotional and/or physiological state are integrated into an individual's surroundings and the devices that the individual makes regular use of. Health monitoring may be then translated into personalized feedback. The feedback may support immediate wellness and long-term disease prevention. By embedding one or more assessment techniques into an individual's surroundings and/or activities, it becomes easier to collect relevant data about the individual's physiological and mental state over significant periods of time. Further, by making assessments through embedded sensors or devices, the individual avoids stigmas and negative perceptions associated with poor health.

Embodiments of the present invention provide a real-time health feedback and mobile coaching system that is responsive to physiological, contextual and/or self-monitored sensors or indicator(s) associated with an individual. The mobile coaching system may help individuals whose lifestyle, physiological characteristics, and emotional reactivity poses a risk for certain diseases, such as depression, headaches, pain disorders, psychiatric concerns, and so forth. Thus, mobile therapies or interventions are triggered by moment-to-moment changes in an individual's physical state and emotional health and are made available when and where they are most needed. A mobile intervention is administered via a mobile device that provides therapeutic feedback to the individual on a real-time basis either right before, during or directly after a situation relevant to emotional and physical health.

Embodiments of the present invention may help to improve self regulation and limit the cumulative toll of unhealthy behaviors and physiological responses. Therapeutic feedback and interventions may support immediate wellness and long-term disease prevention.

Various embodiments of the present invention may be generally directed to a system, apparatus and method for mobile real-time health feedback based on physiological, contextual and/or self-monitored indicators of changes in health states. Other embodiments may be described and claimed.

Various embodiments may comprise one or more elements or components. An element may comprise any structure arranged to perform certain operations. Each element may be implemented as hardware, software, or any combination thereof, as desired for a given set of design parameters or performance constraints. Although an embodiment may be described with a limited number of elements in a certain topology by way of example, the embodiment may include more or less elements in alternate topologies as desired for a given implementation. It is worthy to note that any reference to “one embodiment” or “an embodiment” means that a particular feature, structure, or characteristic described in connection with the embodiment is included in at least one embodiment. The appearances of the phrase “in one embodiment” in various places in the specification are not necessarily all referring to the same embodiment.

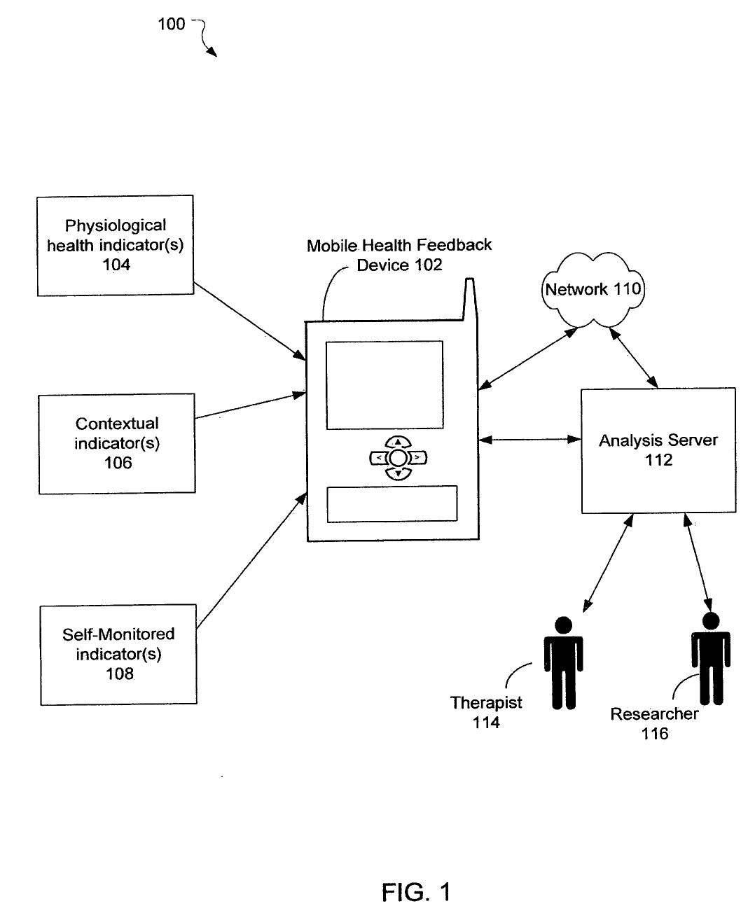

FIG. 1 illustrates one embodiment of a system 100 for mobile real-time health feedback based on physiological, contextual and/or self-monitored indicator(s). In one embodiment, system 100 comprises a mobile health feedback device 102, one or more physiological health indicator(s) 104, one or more contextual indicator(s) 106, one or more self-monitored indicator(s) 108, a network 110, an analysis server 112, a therapist 114 and a researcher 116.

At a high level and in an embodiment, real-time data is continuously collected for an individual via physiological health indicator(s) 104, contextual indicator(s) 106 and/or self-monitored indicator(s) 108. The collected data are transmitted to mobile health feedback device 102. Device 102 processes the data to determine whether the individual is currently in (or about to enter) a potentially unhealthy situation or shift in health state. The data may also be processed by physiological health indicator(s) 104 (e.g., ECG device) and/or by analysis server 112. If a potentially unhealthy situation or shift in health state is detected, mobile health feedback device 102 determines an appropriate intervention. The administered intervention via device 102 provides therapeutic feedback to the individual on a real-time basis either right before, during or directly after this state shift.

Further, data may be collected regarding the effectiveness of the administered intervention and the individual's pattern of reacting to and recovering from certain state shifts and situations. Based on the determined effectiveness of the administered mobile intervention, interventions may be further customized and or adapted for the individual. For example, the invention might determine that an individual's blood pressure is most elevated at 2:00 pm on Monday-Friday while at work and that the individual recovers best from stress most effectively by one type of mobile feedback/intervention at home and another at work.

The collected data and all information stored in device 102 may also be transmitted via network 110 (e.g., the Internet, a local area network (LAN), a wide area network (WAN), etc.) or via a direct connection between device 102 and server 112. All data/information may be communicated via a wireless connection, a wired connection, or some combination of both.

Analysis server 112 may be a back-end server that is used by the invention for more in-depth or historical processing and analysis of the data. Further analysis of the data may also be conducted via therapist 114 and/or researcher 116 and may include patient medical information stored on analysis server 112 or an information system networked to server 112. The results of the more in-depth or historical processing and analysis may be forwarded to mobile health feedback device 102 as a feedback in order to adapt and improve the overall effectiveness of the administered mobile interventions.

Embodiments of the invention may also determine when the individual most effectively self-regulates. Here, positive reinforcement/encouragement is provided to the individual by mobile health feedback device 102. Each of the components or elements of system 100 will be discussed next in more detail.

In various embodiments, system 100 may be implemented as a wireless system, a wired system, or a combination of both. When implemented as a wireless system, system 100 may include components and interfaces suitable for communicating over a wireless shared media, such as one or more antennas, transmitters, receivers, transceivers, amplifiers, filters, control logic, and so forth. An example of wireless shared media may include portions of a wireless spectrum, such as the RF spectrum and so forth. When implemented as a wired system, system 100 may include components and interfaces suitable for communicating over wired communications media, such as input/output (I/O) adapters, physical connectors to connect the I/O adapter with a corresponding wired communications medium, a network interface card (NIC), disc controller, video controller, audio controller, and so forth. Examples of wired communications media may include a wire, cable, metal leads, printed circuit board (PCB), backplane, switch fabric, semiconductor material, twisted-pair wire, co-axial cable, fiber optics, and so forth.

As discussed above, real-time data is continuously collected for an individual via physiological health indicator(s) 104, contextual indicator(s) 106 and/or self-monitored indicator(s) 108. The collected data may be wirelessly transmitted to mobile health feedback device 102 via, for example, Bluetooth technology, Zigbee technology or a proprietary system. The invention is not limited to these example wireless technologies. Alternatively, indicator(s) 104, 106 and/or 108 may transmit data to device 102 via a wired connection, or some combination of wireless and wired connection technologies.

Indicator(s) 104, 106 and/or 108 may also be adapted to store real-time data via integrated long term storage, such as flash memory for example, and then transmit the data to mobile health feedback device 102 at a later time. The integrated long term storage helps to ensure that no collected data are lost if there is no connection currently available with device 102.

An additional benefit of the invention is that the form factors for the monitoring of data and the administration of the mobile therapies are non-stigmatizing. Thus, the invention may especially appeal to individuals who are concerned about health and wellness, but do not want to announce his or her concerns publicly.

In an embodiment of the invention, health monitoring occurs on a small form factor that integrates physiological and activity sensing. Physiological health indicator(s) 104 may be small form factor devices that are worn by the individual and that are capable of monitoring and or measuring a biological activity or function. Physiological health indicator(s) 104, for example, may include a small form factor that combines one or more of a pulse oximeter unit to measure oxygenation level; a multiaxial accelerometer to measure activity level and orientation; and a temperature sensor to measure temperature level. The integrated physiological health monitoring may also include one or more of a unit to measure galvanic skin response; a pulse wave velocity monitor to monitor blood pressure; a minimally invasive or noninvasive glucometry monitor unit to measure blood sugar; a unit to measure spirometry; a unit to measure respiration rate; a unit to measure speech characteristics (including volume, characteristics of speech associated with stress, speech cadence, latency, pause length, phonetic distinctness, word transposition, speech prosody, and indications of affect and variability in these speech characteristics); a unit to measure typing speed and errors; a unit to measure hormonal levels (e.g., an EMG device); a unit to measure caloric expenditure; a unit to measure temperature levels; a unit to measure muscle tension (e.g., cortisol); and so forth. One or more of these indicators or units may be used either individually or in combination to receive real-time health feedback about an individual. These examples are not meant to limit the invention. In fact, the invention contemplates the use of any means to monitor an individual.

The integrated physiological monitoring is able to identify physical and emotional health indicators with higher accuracy than is possible with any individual sensor. For example, an increase in skin temperature following activity may be a normal physiological response, but without activity may indicate illness.

In an embodiment of the invention, physiological health indicator(s) 104 is an integrated physiological monitor worn by an individual as a wireless chest worn sensor. The sensor may communicate with mobile health feedback device 102 via a Body Area Network (BAN)—a short-range wireless network to transmit monitored data.

In an embodiment of the invention, contextual indicator(s) 106 may include location sensors in the individual's environment to indicate arrival at places associated with changes in health states. For example, location sensors may be placed in the individual's car, workplace, home, gym, and social environments and may interact with identification sensors that are worn and/or incorporated into mobile health feedback device 102, and so forth. Location information may also be obtained via Global Positioning System (GPS) technology.

Contextual indicator(s) 106 may also include a calendar sync with mobile health feedback device 102 to indicate upcoming events associated with changes in physical and emotional health states (e.g., hormonal cycles, seasonal changes, changes in work patterns, first time meeting future in-laws, job interview appointments, etc.) and/or times during the day associated with stress (e.g., daily drive to and from work during rush hour, etc.).

In embodiments of the invention, self-monitored indicator(s) 108 may include various ways in which an individual may provide data or feedback to the mobile health device 102 via direct or indirect input into device 102. This may include, but is not necessarily limited to, an array of health journaling techniques, such as menu selection of adjectives to indicate emotional and physical health states, social context and behaviors (e.g. eating, exercise, sleeping) menu selection of dietary intake, camera documentation of context, the “panic button” means of initiating the mobile intervention, selection of images to represent health state or situation, etc. Items can be selected via touch screen, navigation buttons, a scroll dial or a stylus, for example.

This health journaling or self-report data is gathered via a method of experience time sampling (ETS) in which the device prompts users for responses at frequent time intervals (for example every 30 minutes). Experience time sampling allows researchers to assess health states and behavior frequently and very close in time/proximal to events in daily life, rather than asking people to report retrospectively. Many studies have demonstrated extreme inaccuracy in retrospective self-report. The intuitive, less intrusive and more expedient translation of questionnaires for a mobile interface (e.g., device 102) described herein are more suitable for frequent administration and therefore facilitate frequent health surveying (e.g., ETS). Experience sampling can also be triggered by contextual factors (stressful appointments or meetings, entered manually or automatically detected via synchronization with an online calendar, etc.), location (via beacons, GPS, etc.), time of day (morning and evening journaling), or upon completion of mobile therapy interventions (asking mood after mobile interventions).

In embodiments of the invention, journaling refers to all types of input. For example, journaling allows the individual to report on his or her health states and related behaviors through a variety of modalities including responses to questionnaires, touching of iconic images, gesturing, menu selection, touch screen activation, and spoken input. The capture of voice notes via journaling allows for analysis of health indicator(s) within the content and quality of the user's speech. For example, speech volume, clarity, precision and slippages may be indicators of depression, cognitive decline, insomnia, medication side-effects or other health concerns.

As discussed above, mobile health feedback device 102 receives real-time (or stored) data via physiological health indicator(s) 104, contextual indicator(s) 106 and/or self-monitored indicator(s) 108. Device 102 processes the data to determine whether the individual is currently in (or about to enter) a potentially unhealthy state or situation. The data may also be processed by physiological health indicator(s) 104 (e.g., ECG device) and/or by analysis server 112. If a potentially unhealthy situation or shift in health state is detected, mobile health feedback device 102 determines an appropriate mobile intervention. The administered mobile intervention via device 102 provides therapeutic feedback to the individual on a real-time basis either before, during or directly after the event.

In one embodiment, mobile health feedback device 102 may be any mobile device capable of performing the functionality of the invention described herein. Device 102 may be implemented as part of a wired communication system, a wireless communication system, or a combination of both. In one embodiment, for example, device 102 may be implemented as a mobile computing device having wireless capabilities. A mobile computing device may refer to any device having a processing system and a mobile power source or supply, such as one or more batteries, for example.

Examples of embodiments of a mobile computing device that may be adapted to include the functionality of the present invention include a laptop computer, ultra-laptop computer, portable computer, handheld computer, palmtop computer, personal digital assistant (PDA), cellular telephone, combination cellular telephone/PDA, smart phone, pager, one-way pager, two-way pager, messaging device, data communication device, and so forth.

Examples of such a mobile computing device also may include computers that are arranged to be worn by a person, such as a wrist computer, finger computer, ring computer, eyeglass computer, belt-clip computer, arm-band computer, shoe computers, clothing computers, and other wearable computers.

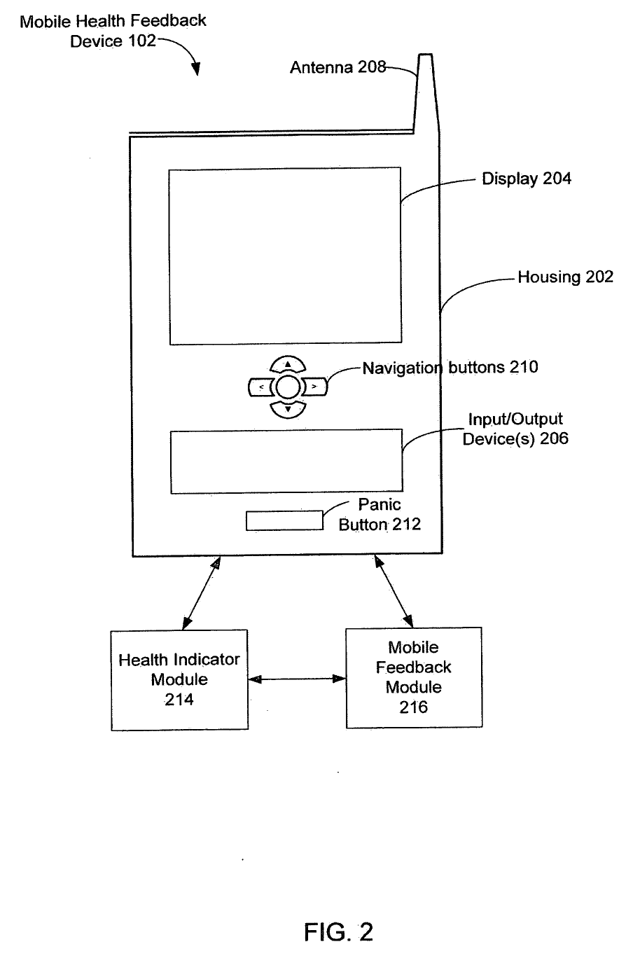

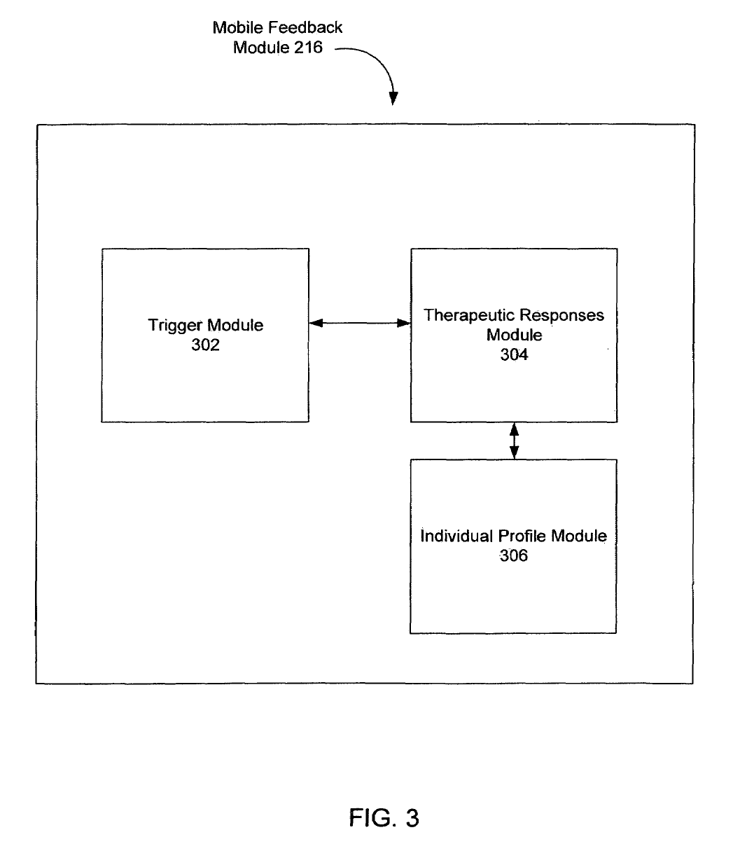

A more detailed description of an embodiment of mobile therapy device 102 is shown in FIGS. 2 and 3. Referring to FIG. 2, device 102 may include a housing 202, a display 204, one or more input/output devices 206, an antenna 208, navigation buttons 210, a panic button 212, a health indicator module 214 and a mobile feedback module 216.

Health indicator module 214 and mobile feedback module 216 may be directly integrated into device 102 or may be coupled to device 102 via a connection (e.g., wireless, wired or some combination of both). Note that although the functionality of modules 214 and 216 is described herein as being separated into two components, this is not meant to limit the invention. In fact, this functionality may be combined into one component or separated into three for more components. Additionally, one or both of health indicator module 214 and mobile feedback module 216 may be customized for an individual. Each of the components of FIG. 2 is described next in more detail.

Housing 202 may comprise any suitable housing, but typically involves a small form factor to enable mobile health feedback device 102 to be easily transportable.

Display 204 may comprise any suitable display unit for displaying information appropriate for a mobile computing device. Display 204 is used by the invention to display mobile interventions to the individual, to assist with input into device 102, and so forth.

I/O device(s) 206 may comprise any suitable I/O device for entering information into and receiving information from mobile computing device 102. In embodiments of the invention, input is gathered implicitly from physiological monitoring and via touching iconic images on a screen to indicate ratings, for example. Input may also be gathered by gestures (e.g., turning mobile health feedback device 102 upside down to indicate state of mind, etc.).

Examples for I/O device(s) 206 may include touch screen interfaces, simple menus with icon selection, gestural manipulation of the device, a suitable alphanumeric keyboard, a numeric keypad, a touch pad, input keys, buttons, switches, rocker switches, a microphone, a speaker, voice recognition device and software, as well as all of the physiological sensing described above, and so forth. Information may be entered into device 102 by way of microphone. Such information may be digitized by a voice recognition device. The embodiments are not limited in this context.

Antenna 208 is used to facilitate wireless communication with mobile health feedback device 102.

In one embodiment, navigation buttons 210 comprise an upward navigation button, a downward navigation button, a leftward navigation button, and a rightward navigation button. Navigation buttons 210 also may comprise a select button to execute a particular function on mobile health feedback device 102.

Mobile interventions can be initiated by the user in several ways. For example, a menu allows quick access to breathing and relaxation exercises and a “panic button” (e.g., panic button 212) prompts a phone call that provides the user with a socially acceptable excuse to leave a negative situation.

As described above, health indicator module 214 processes the data sent from physiological health indicator(s) 104, contextual indicator(s) 106 and/or self-monitored indicator(s) 108 to determine whether the individual is currently in (or about to enter) a different health state or situation. If so, mobile feedback module 216 determines an appropriate mobile intervention. The administered mobile intervention via device 102 provides therapeutic feedback to the individual on a real-time basis before, during or directly after the state or situational shift.

FIG. 3 illustrates one embodiment of mobile feedback module 216. Referring to FIG. 3, module 216 may comprise a trigger module 302, a therapeutic responses module 304 and an individual profile module 306. Note that although the functionality of modules 302, 304 and 306 is described herein as being separated into three components, this is not meant to limit the invention. In fact, this functionality may be combined into one or two components, or separated into four or more components. Trigger module 302, therapeutic responses module 304 and individual profile module 306 all may be customized to an individual, as will be described in more detail below.

Trigger module 302 processes the output of health indicator module 214 (FIG. 2) to determine one or more possible interventions to administer on mobile device 102. For example, assume that the output of health indicator module 214 indicates that the individual's health indicators are changing while she is driving. Note that the current situation of the individual may have been determined via data received from physiological health indicator(s) 104 and location sensors of contextual indicator(s) 106. Here, so as to not impair the driving ability of the individual, trigger module 302 may determine that an audio intervention such as music is more appropriate than a visual intervention such as images, text or graphs.

As discussed above, a mobile intervention is supportive feedback administered via a mobile device on a real-time basis either right before, during or directly after a change in health state or a health related situation. One or more mobile therapies may be defined and stored in therapeutic responses module 304.

Mobile interventions may include, but are not limited to, interventions such as biofeedback, breathing exercises, progressive muscle relaxation exercises, timely presentation of personal media (e.g., music and images collected from users), offers of an exit strategy (e.g., a phone call that helps the user escape from an unhealthy situation), references to a range of psychotherapeutic techniques and graphical representations of trends (e.g., depictions of health metrics over time), visual (e.g., picture), delay tactics (e.g., hourglass animation), breathing exercises, audio (e.g., music or humorous quotes), biofeedback, cognitive reframing, progressive muscle relaxation, visual trend analysis, and so forth. These example mobile interventions are provided for illustration purposes only and are not meant to limit the invention.

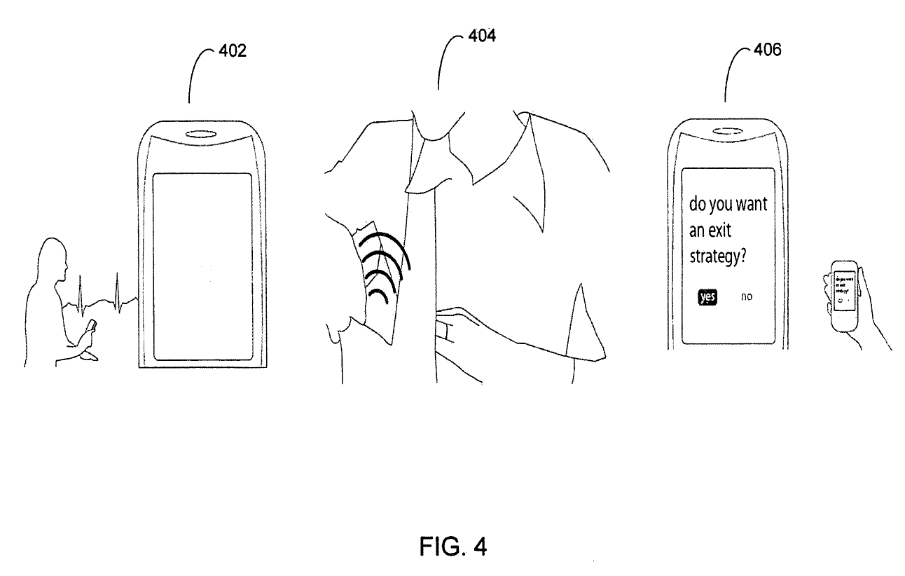

FIG. 4 illustrates an embodiment of an exit strategy intervention administered by mobile health feedback device 102. Referring to FIG. 4, picture 402 illustrates a woman being monitored by an integrated physiological monitor (of physiological health indicator(s) 104). The integrated physiological monitor is sending data to mobile health feedback device 102 that indicates the woman feels stressed.

In picture 404 of FIG. 4, mobile health feedback device 102 provides a signal to the woman (e.g., audible signal, vibration, etc.). In picture 406, device 102 displays a text message that asks the woman if she wants an “exit strategy.” The exit strategy may include a phone ring or a vibration administered via device 102 that allows the woman to excuse herself from the stressful situation. The example illustrated in FIG. 4 is provided for illustration purposes only and is not meant to limit the invention.

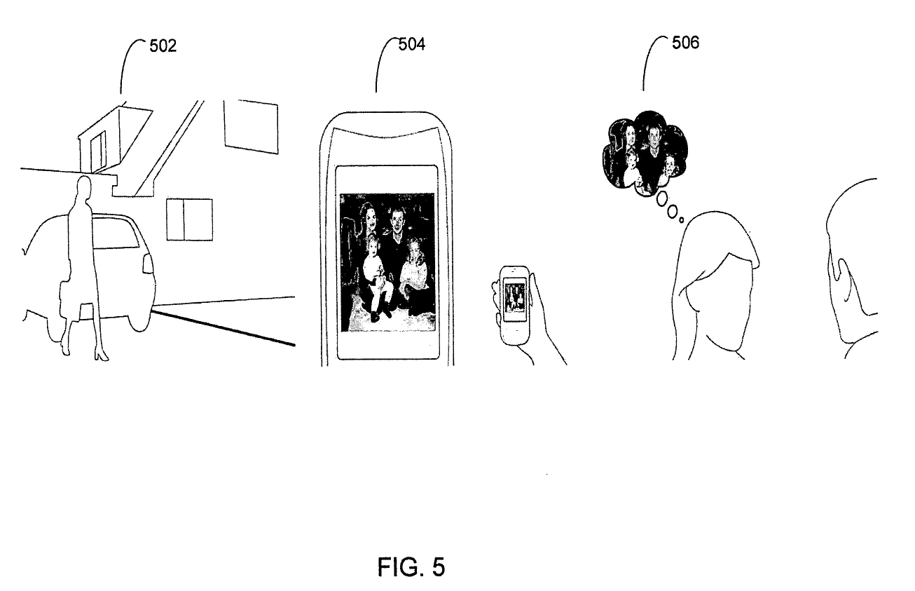

FIG. 5 illustrates an embodiment of picture therapy administered by mobile health feedback device 102. Referring to FIG. 5, picture 502 illustrates a woman rushing home after work to pick up her husband. Location sensing technologies (e.g., GPS technology) allow device 102 to mark geographical transitions and tailor the mobile intervention accordingly.

In picture 504 of FIG. 5, mobile health feedback device 102 has processed the received data and determines that the woman is experiencing a health shift. The health shift is likely the result of challenging events involved in the transition from work to home (e.g., driving through rush hour traffic). Mobile health feedback device 102 prepares the woman for her transition from work to home by beaming to her a photo of her family. The picture helps the woman cope with the challenges of getting home.

In picture 506 of FIG. 5, the woman gets bad news from her husband that the babysitter has canceled. The woman keeps the happy picture in her mind and is able to remain calm. The example illustrated in FIG. 5 is provided for illustration purposes only and is not meant to limit the invention.

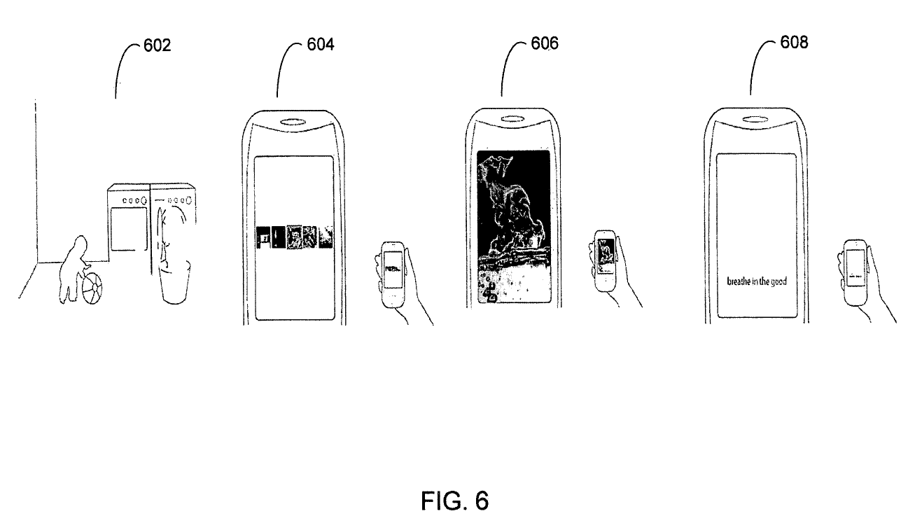

FIG. 6 illustrates an embodiment of a breathing exercise intervention administered by mobile health feedback device 102. Referring to FIG. 6, picture 602 illustrates a woman multitasking around the house and her child has just opened the washing machine causing water and suds to overflow. In picture 604 of FIG. 6, the woman hits the “panic button.” In picture 606, mobile health feedback device 102 registers rage. In picture 608, device 102 administers breathing exercise intervention on device 102. The example illustrated in FIG. 6 is provided for illustration purposes only and is not meant to limit the invention.

Audio interventions involve a clip of music or humorous quotes being played for the individual via mobile health feedback device 102. The clips of music or humorous quotes are meant to relax the individual.

A range of mobile interventions help the user delay gratification of problematic behaviors. One example is an hourglass animation that could help someone to wait a certain length of time before submitting to a cigarette craving, eating a donut or even the temptation to get involved in a destructive confrontation. Often by waiting a short while, people can realize that they don't really need the cigarette, donut, or to engage in other problematic behavior.

Biofeedback displays on mobile health feedback device 102 (e.g., a phone) raise self-awareness and mindfulness about health by showing an individual immediate data on physiological functioning (from the indicator(s)).

Cognitive reframing is a process in which individuals critically evaluate automatic thoughts and interpretations that are maladaptive. Cognitive reframing involves practices that the individual is learning in a clinical or self-help setting. Here, the mobile application (“mind scan”) reminds the user to question their interpretations.

Progressive muscle relaxation is a therapeutic technique in which individuals tense and relax different areas of the body—one at a time. For example, a phone display (“body scan”) walks the individual through the major muscle groups and assists in the tensing and relaxation of relevant muscles.

A presentation of visual trends and other analysis allows individual to view patterns of physical and emotional health indicators over time and their correlation with contextual factors. Visual stimuli are displayed on mobile health feedback device 102. Clinicians can view these during and before treatment to modulate treatment and monitor patients. Queries on the database allow for different visual trends to be presented to the individual or clinician.

Referring back to FIG. 3, information stored in therapeutic responses module 304 for each intervention may include variations of the intervention itself, like escalations based on whether or not the individual responds physiologically to a given therapy, analogous to dosing or titrating of medication where the amount of medicine is tailored. Escalations in modality may also occur to encourage interaction with the system. For example, if the patient doesn't respond to a glowing of the phone display, the phone may vibrate and eventually chime. The broad range of variations to the mobile interventions are all assumed to be within the scope of the present invention.

In an embodiment of the invention, these variations of the mobile therapies or interventions may be set as determined by the medical profession regarding what is effective for certain health concerns and related situations.

In other embodiments, the variations of the mobile therapies may be adapted or customized for an individual according to what the present invention determines has been effective for the individual in the past to recover from changes in health states and related situations. For example, the invention might determine that an individual is most fatigued at 2:00 pm on Monday-Friday while at work and that the individual recovers best from fatigue most effectively by biofeedback at home and image based interventions at work.

In other embodiments, the invention might determine that the interventions that were most effective in improving health and self-awareness for a particular individual in the past is not working as well to date. Thus, the invention adapts and uses a different type of mobile intervention to better reduce stress in the individual.

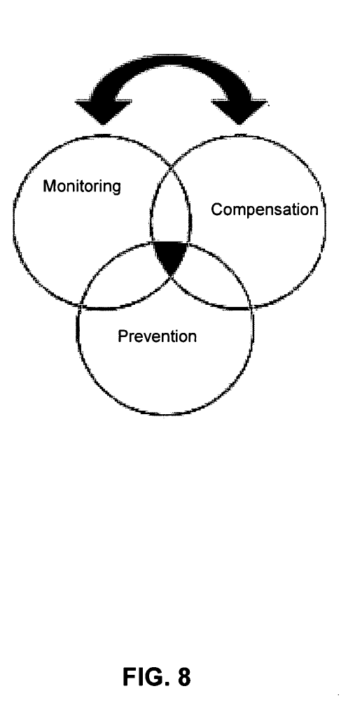

As discussed above, embodiments of the present invention provide for adaptive learning and embedded assessment. In embodiments, the invention tracks the individual's trends in physiological patterns that indicate improved or worsened handling of health states based on changes in physiological stress as detected by the system. This allows resetting the baseline. This resetting of the patient's baseline is based on principles of adaptive learning. Principles of embedded assessment allow recalibration of feedback based on adaptive learning. See FIG. 8 for the dynamic relationship between continuous monitoring and feedback in the embedded assessment approach.

In other embodiments, the system personalizes interventions by incorporating personal information that is used by the invention and stored in individual profile module 306. For example, the individual may provide his or her own pictures, music clips, humorous quotes, mantras, timeframe for daily commutes, working hours and sleeping patterns, social support network, and so forth. Then, for example, when it is determined that audio intervention should be administered for an individual, the audio presented may be a music clip provided by the individual and stored in module 306 (versus one of the default music clips in module 304).

Operations for the above embodiments may be further described with reference to the following figures and accompanying examples. Some of the figures may include a logic flow. Although such figures presented herein may include a particular logic flow, it can be appreciated that the logic flow merely provides an example of how the general functionality as described herein can be implemented. Further, the given logic flow does not necessarily have to be executed in the order presented unless otherwise indicated. In addition, the given logic flow may be implemented by a hardware element, a software element executed by a processor, or any combination thereof.

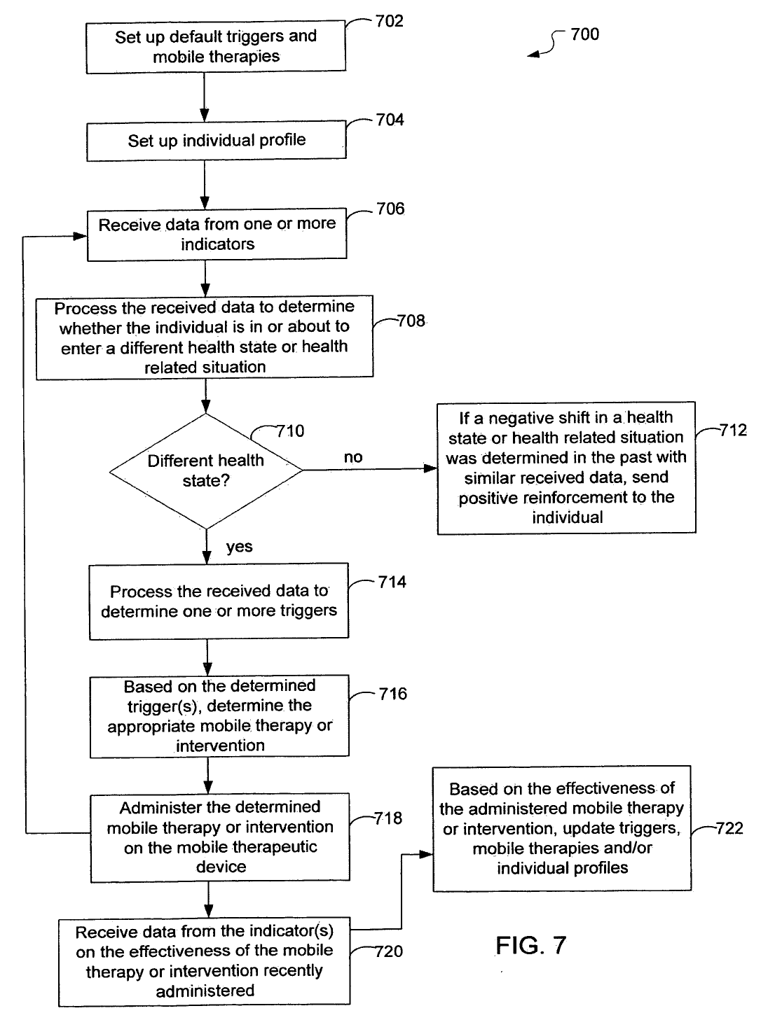

FIG. 7 illustrates one embodiment of a logic flow 700. The logic flow 700 may be representative of the operations executed by one or more embodiments described herein, for example, the operations executed by system 100.

Referring to FIG. 7, at block 702, initial default triggers and mobile interventions are set up in mobile health feedback device 102. For example, default music clips may be defined for audio intervention, default pictures may be defined for visual intervention, and so forth.

At block 704, an individual's profile is set up. This may include, but is not limited to, information provided by the individual. Such information may include, but is not necessarily limited to, pictures, music clips, humorous quotes, mantras, timeframe for daily commutes, working hours and sleeping patterns, social support network, and so forth.

At block 706, data is received by mobile health feedback device 102. In an embodiment, the data received represents data collected about the individual via physiological health indicator(s) 104, contextual indicator(s) 106 and/or self-monitored indicators(s) 108, as described above.

At block 708, the received data is processed by mobile health feedback device 102 to determine whether the individual is in (or about to enter) a different health state or health related situation. The data may also be processed by physiological health indicator(s) 104 (e.g., ECG device) and/or by analysis server 112.

At block 710, if it is determined that the individual is not currently in a potentially health deleterious situation, control passes to block 712. The invention is adapted to determine when the individual handles a health event better in the present than he or she did in the past. In block 712, it is determined whether a potentially health deleterious situation was determined in the past with similar received data for the individual. If so, then positive reinforcement/encouragement is provided to the individual by mobile health feedback device 102 for handling the health deleterious situation better now than in the past.

At block 710, if it is determined that the individual is currently in (or about to enter) a potentially health deleterious situation, control passes to block 714. In block 714, the received data is processed by mobile health feedback device 102 to determine one or more triggers. As discussed above, triggers indicate one or more possible mobile interventions to administer on mobile health feedback device 102.

At block 716, based on the determined triggers, mobile health feedback device 102 determines the appropriate mobile intervention to administer. At block 718, the determined mobile intervention is administered on mobile health feedback device 102.

Control then goes back to block 706, where the individual is continuously monitored via physiological health indicator(s) 104, contextual indicator(s) 106 and/or self-monitored indicator(s) 108.

At block 720, since the individual is continuously monitored, mobile health feedback device 102 may analyze the health status of the individual immediately after the administered mobile intervention to determine its effectiveness.

At block 722, data stored in mobile health feedback device 102 and data stored in analysis server 112 may be updated to reflect the effectiveness of the administered mobile intervention.

Various embodiments may be implemented using hardware elements, software elements, or a combination of both. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Some embodiments may be implemented, for example, using a machine-readable or computer-readable medium or article which may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

Numerous specific details have been set forth herein to provide a thorough understanding of the embodiments. It will be understood by those skilled in the art, however, that the embodiments may be practiced without these specific details. In other instances, well-known operations, components and circuits have not been described in detail so as not to obscure the embodiments. It can be appreciated that the specific structural and functional details disclosed herein may be representative and do not necessarily limit the scope of the embodiments.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. An apparatus comprising:
a processor, a mobile power supply, at least one input device, and at least one output device; and
wherein the apparatus is capable of receiving data relating to the physical or mental health states of a user from an input device and passive sensing, wherein the apparatus is capable of providing a mobile intervention via the at least one output device, and wherein the apparatus is capable of measuring the effectiveness of the mobile intervention in improving the physical or mental health states and generating self-awareness.

2. The apparatus of claim 1, wherein the apparatus is additionally capable of selecting the mobile intervention based on a previous effectiveness measurement.

3. The apparatus of claim 1, wherein the input device is a physiological health sensor that comprises a small form factor of integrated physiological and activity monitoring of the user.

4. The apparatus of claim 3, wherein the integrated physiological and activity monitoring includes monitoring of at least one of oxygen level, activity levels, and temperature levels.

5. The apparatus of claim 4, wherein the integrated physiological and activity monitoring further includes monitoring of at least one of galvanic skin response, blood pressure, blood sugar, spirometry, respiration rate, speech characteristics, typing speed and errors, hormonal levels, caloric expenditure, and muscle tension.

6. The apparatus of claim 1, wherein the input device is a contextual indicator that comprises at least one of location sensors and a calendar sync function.

7. The apparatus of claim 1, wherein the input device is a self-monitored indicator that comprises at least one of experience time sampling of mood and physical well being and journaling, wherein journaling allows the individual to report on his or her health states and related behaviors through one or more of responses to questionnaires, touching of iconic images, gesturing, menu selection, touch screen activation, and spoken input.

8. The apparatus of claim 7, wherein the experience time sampling includes prompting the user for responses at frequent time intervals.

9. The apparatus of claim 7, wherein the experience time sampling may be triggered by one or more of location, a calendar sync function, time of day and upon completion of the mobile intervention.

10. The apparatus of claim 1, wherein the mobile intervention comprises at least one of biofeedback, breathing exercises, progressive muscle relaxation exercises, timely presentation of personal media, offers of an exit strategy, references to a range of psychotherapeutic techniques and graphical representations of trends, visual displays, delay tactics, audio, cognitive reframing, progressive muscle relaxation and visual trend analysis.

11. The apparatus of claim 1, wherein the mobile intervention is provided to the user either before, during or immediately after a change in health states.

12. The apparatus of claim 1, wherein the input device communicates wirelessly with the apparatus.

13. A method comprising:
receiving data relating to the physical or mental health states of a user from an input device and passive sensing;
providing a mobile intervention via at least one output device; and
measuring the effectiveness of the mobile intervention in improving the physical or mental health states and generating self-awareness.

14. The method of claim 13, further comprising selecting the mobile intervention based on a previous effectiveness measurement.

15. The method of claim 13, wherein the input device is a physiological health sensor that comprises a small form factor of integrated physiological and activity monitoring of the user.

16. The method of claim 15, wherein the integrated physiological and activity monitoring includes monitoring of at least one of oxygen level, activity levels, and temperature levels.

17. The method of claim 16, wherein the integrated physiological and activity monitoring further includes monitoring of at least one of galvanic skin response, blood pressure, blood sugar, spirometry, respiration rate, speech characteristics, typing speed and errors, hormonal levels, caloric expenditure, and muscle tension.

18. The method of claim 13, wherein the input device is a contextual indicator that comprises at least one of location sensors and a calendar sync function.

19. The method of claim 13, wherein the input device is a self-monitored indicator that comprises at least one of experience time sampling of mood and physical well being and journaling, wherein journaling allows the individual to report on his or her health states and related behaviors through one or more of responses to questionnaires, touching of iconic images, gesturing, menu selection, touch screen activation, and spoken input.

20. The method of claim 19, wherein the experience time sampling includes prompting the user for responses at frequent time intervals.

21. The method of claim 19, wherein the experience time sampling may be triggered by one or more of location, a calendar sync function, time of day and upon completion of the mobile intervention.

22. The method of claim 13, wherein the mobile intervention comprises at least one of biofeedback, breathing exercises, progressive muscle relaxation exercises, timely presentation of personal media, offers of an exit strategy, references to a range of psychotherapeutic techniques and graphical representations of trends, visual displays, delay tactics, audio, cognitive reframing, progressive muscle relaxation and visual trend analysis.

23. The method of claim 13, wherein the mobile intervention is provided to the user either before, during or immediately after a change in health states.

24. The method of claim 13, wherein the input device is a wireless device.

25. A machine-readable medium containing instructions which, when executed by a processing system, cause the processing system to perform instructions for:
receiving data relating to the physical or mental health states of a user from an input device and passive sensing;
providing a mobile intervention via at least one output device; and
measuring the effectiveness of the mobile intervention in improving the physical or mental health states and generating self-awareness.

26. The machine-readable medium of claim 25, further comprising selecting the mobile intervention based on a previous effectiveness measurement.

27. The machine-readable medium of claim 25, wherein the input device is a physiological health sensor that comprises a small form factor of integrated physiological and activity monitoring of the user.

28. The machine-readable medium of claim 27, wherein the integrated physiological and activity monitoring includes monitoring of at least one of oxygen level, activity levels, and temperature levels.

29. The machine-readable medium of claim 28, wherein the integrated physiological and activity monitoring further includes monitoring of at least one of galvanic skin response, blood pressure, blood sugar, spirometry, respiration rate, speech characteristics, typing speed and errors, hormonal levels, caloric expenditure, and muscle tension.

30. The machine-readable medium of claim 25, wherein the input device is a contextual indicator that comprises at least one of location sensors and a calendar sync function.

31. The machine-readable medium of claim 25, wherein the input device is a self-monitored indicator that comprises at least one of experience time sampling of mood and physical well being and journaling, wherein journaling allows the individual to report on his or her health states and related behaviors through one or more of responses to questionnaires, touching of iconic images, gesturing, menu selection, touch screen activation, and spoken input.

32. The machine-readable medium of claim 25, wherein the experience time sampling includes prompting the user for responses at frequent time intervals.

33. The machine-readable medium of claim 25, wherein the experience time sampling may be triggered by one or more of location, a calendar sync function, time of day and upon completion of the mobile intervention.

34. The machine-readable medium of claim 25, wherein the mobile intervention comprises at least one of biofeedback, breathing exercises, progressive muscle relaxation exercises, timely presentation of personal media, offers of an exit strategy, references to a range of psychotherapeutic techniques and graphical representations of trends, visual displays, delay tactics, audio, cognitive reframing, progressive muscle relaxation and visual trend analysis.

35. The machine-readable medium of claim 25, wherein the mobile intervention is provided to the user either before, during or immediately after a change in health states.

36. The machine-readable medium of claim 25, wherein the input device is a wireless device.
